(19) United States
(12) Patent Application Publication
(10) Pub. No.: US 2007/0249822 A1
(43) Pub. Date: Oct. 25, 2007

(54) Photolabile Coumarinylmethyl Esters of Cyclic Nucleotides, Methods for their Preparation and Their Use

(75) Inventors: Volker Hagen, Berlin (DE); Ulrich Benjamin Kaupp, Aachen (DE); Jurgen Bendig, Berlin (DE); Burghard Wiesner, Berlin (DE)

Correspondence Address: NORRIS, MCLAUGHLIN & MARCUS, P.A., 875 THIRD AVE, 18TH FLOOR, NEW YORK, NY, 10022, US

(73) Assignees: Forschungsverbund Berlin E.V., Berlin (DE); Forschungszentrum Julich Gmbh, Julich (DE)

(21) Appl. No.: 10/257,793

(22) PCT Filed: Mar. 26, 2001
(86) PCT No.: PCT/EP01/03512

§ 371 Date: Jun. 3, 2003

(30) Foreign Application Priority Data
Apr. 20, 2000 (DE) 10021256.5

(51) Int. Cl. C07H 19/16

(52) U.S. Cl. 536/27.12; 536/27.2

(57) ABSTRACT

The invention relates to new photolabile coumarinylmethyl esters of cyclic nucleotides of general formula I 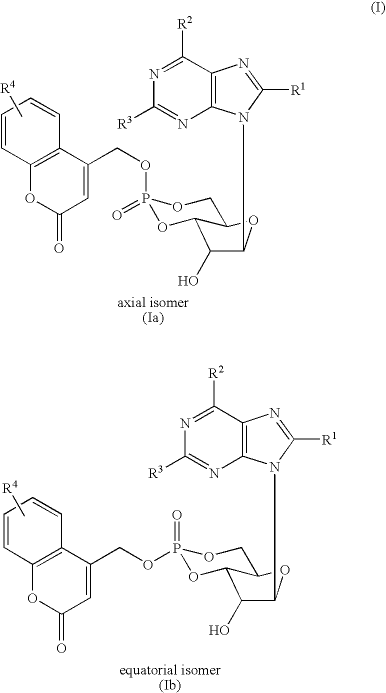 wherein
R¹ represents hydrogen, bromine or p-chlorophenylthio
R⁴ represents 7-carboxymethoxy, 6,7-, 5,7- or 7,8-bis-(carboxymethoxy), 7-C₁-C₃-alkoxycarbonylmethoxy, 6,7-bis(C₁-C₃-alkoxycarbonylmethoxy), 5,7-bis(C₁-C₃-alkoxy-carbonylmethoxy), or 7,8-bis(C₁-C₃-alkoxycarbonylmethoxy),
R² represents —NH₂ and R³ is hydrogen (adenine residue), or
R² represents —OH and R³ represents NH₂ (guanine residue) The inventive compounds are biologically inactive and exhibit high solubility in aqueous buffer solutions, high efficiency in photocleavage and rapid kinetics of liberation, together with high resistance to solvolysis.

The invention relates to new photolabile coumarinylmethyl esters of cyclic nucleotides of general formula I 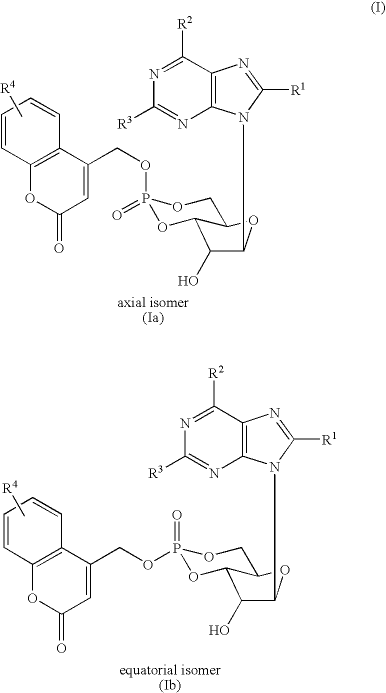
wherein
R¹ represents hydrogen, bromine or p-chlorophenylthio,
R⁴ represents 7-carboxymethoxy, 6,7-, 5,7- or 7,8-bis-(carboxymethoxy), 7-C₁-C₃-alkoxycarbonylmethoxy, 6,7-bis(C₁-C₃-alkoxycarbonylmethoxy), 5,7-bis(C₁-C₃-alkoxy-carbonylmethoxy), or 7,8-bis(C₁-C₃-alkoxycarbonylmethoxy-oxy),
R² represents —NH₂ and R³ is hydrogen (adenine residue), or
R² represents —OH and R³ represents —NH₂ (guanine residue).

Furthermore, the invention relates to a method of producing compounds of formula I and to their use as biologically inactive, photolabile precursors in the photochemical liberation of the corresponding biologically active cyclic nucleotides to investigate cyclic nucleotide-dependent cellular processes in biological systems.

Nucleotides such as cyclic guanosine 3′,5′-monophosphate (cGMP) and cyclic adenosine 3′,5′-monophosphate (cAMP) are known to control a variety of important cellular processes. In order to influence and investigate such cyclic nucleotide-dependent processes with regard to time and mechanistically, chemical derivatives of these nucleotides (so-called “caged” compounds), which are required to be biologically inactive and photolabile, are incorporated in biological systems and converted to the biologically active cyclic nucleotides by irradiation with light. As a rule, cell lines are used as biological systems in such investigations.

The caged compounds employed are to meet high demands. They should have sufficiently high solubility in aqueous buffer solutions, rapid photochemical reactions proceeding with high efficiency, high absorbance at wavelengths >300 nm, lowest possible sensitivity to hydrolysis, low toxicity, and no relevant biological activity. Quite a number of photolabile cyclic nucleotide esters have been described in the literature so far, but they all involve various draw-backs. Also, the data available at present are not sufficient to allow a priori predictions as to the properties of new caged compounds and, in addition, there is always the possibility of undesirable pre-photolytic biological activity when used on cellular systems [K. R. Gee et al., Bio-chemistry 38, 3140 (1999)].

To date, the 2-nitrobenzyl, 4,5-dimethoxy-2-nitrobenzyl and 1-(2-nitrophenyl)ethyl esters of cyclic guanosine 3′,5′-monophosphate (cGMP) and of cyclic adenosine 3′,5′-mono-phosphate (cAMP), as well as the (7-methoxycoumarin-4-yl)-methyl ester of cAMP [T. Furuta et al., J. Org. Chemistry 60, 3953 (1995)], the 7-hydroxy- and other acyloxycoumarinylmethyl esters of cAMP [T. Furuta et al., Methods in Enzymology, Vol. 291 (Ed.: G. Marriott), 50, (1998), T. Furuta et. al., Biochem. Biophys. Res. Commun. 228, 193 (1996)] have been described in the literature. The 4,5-dimethoxy-2-nitrobenzyl, 1-(2-nitrophenyl)ethyl and (7-methoxycoumarin-4-yl)methyl esters of 8-Br- or 8-p-chlorophenylthio-cAMP and 8-Br- or 8-p-chlorophenylthio-cGMP [WO 97/05155; V. Hagen et al., Biochemistry, 35, 7762 (1996); V. Hagen et al., J. Photochem. Photobiol. B: Biol. 42, 71 (1998); V. Hagen et al., C. Photochem. Photobiol. B: Biol. 53, 91 (1999)], and the 4,5-bis(carboxymethoxy)-2-nitrobenzyl ester of cGMP [V. Lev-Ram et al., Neuron 18, 1025 (1997)] have also been used.

Also, the (7-methoxycoumarin-4-yl)methyl ester of CGMP [B. Schade et. al, J. Org. Chem. 64, 9109 (1999)], the desyl ester of cAMP [R. S. Givens et. al, J. Am. Chem. Soc. 114, 8708 (1992)], the (anthraquinon-2-yl)methyl and the (naphth-2-yl)methyl ester of cAMP [T. Furuta et al., J. Org. Chem. 60, 3953 (1995)] have been described but apparently not used as caged compounds in biological experiments so far.

All of the photolabile cyclic nucleotide esters described to date involve a number of drawbacks.

Due to esterification of the free phosphoric acid group, the solubility of the described caged cyclic nucleotides in aqueous buffer solutions is normally limited.

Moreover, the 4,5-dimethoxynitrobenzyl esters of the cyclic nucleotides cAMP and cGMP and of their derivatives exhibit relatively low quantum yields in photolysis, low rate of photolysis and, in addition, the equatorial isomers in particular are sensitive to solvolysis in aqueous buffer solutions [V. Hagen et al., Biochemistry 35, 7762 (1996)]. To date, no physicochemical data relating to the 4,5-bis(carboxymethoxy)-2-nitrobenzyl ester of CGMP are available. Considering the mechanism of photolysis or solvolysis which also applies to the above compounds, it must be assumed that this ester would exhibit analogous disadvantageous properties.

The 1-(2-nitrophenyl)ethyl esters of the cyclic nucleotides utilized so far involve the drawback of sluggish liberation kinetics and low absorption at wavelengths of more than 300 nm. Low absorption, as well as low quantum yield of photolysis, result in unsatisfactory photolysis efficiency which, on the one hand, can be compensated by irradiation higher in energy or by longer periods of exposure which, on the other hand, normally give rise to cell lesions.

The desyl ester of cAMP is sensitive to solvolysis in aqueous buffer solutions, and the (anthraquinon-2-yl)methyl and (naphth-2-yl)methyl esters of cAMP have insufficient solubility in aqueous buffer solutions so that these compounds in many issues are unsuitable as phototriggers for cAMP. The use of the (7-methoxycoumarin-4-yl)methyl esters of cAMP and cGMP or of 8-Br-cAMP or 8-Br-cGMP is also severely restricted as a result of the relatively low solubility in aqueous buffer solutions. Moreover, the equatorial isomer of the (7-methoxycoumarin-4-yl)methyl ester of cAMP has been described to have a relatively low hydrolytic halflife (60 hours) in Ringer's solution.

The 7-hydroxy- and 7-acyloxycoumarinylmethyl esters of cAMP have also been described to have low hydrolytic halflife, and the photochemical quantum yields are only half as high as those of the 7-methoxycoumarinylmethyl ester. Furthermore, these compounds might be expected to have limited solubility.

It was the object of the present invention to provide new photolabile cyclic nucleotide derivatives which should have high solubility in aqueous buffer solutions, as well as high efficiency in photocleavage and preferably rapid kinetics of liberation, together with high resistance to solvolysis. Importantly, the compounds should not have any pre-photolytic biological activity so as to be usable in investigating cellular systems.

The object of the invention is accomplished by means of new coumarinylmethyl esters of cyclic guanosine 3′,5′-mono-phosphate (cGMP) and cyclic adenosine 3′,5′-monophosphate (cAMP) according to the general formula I, 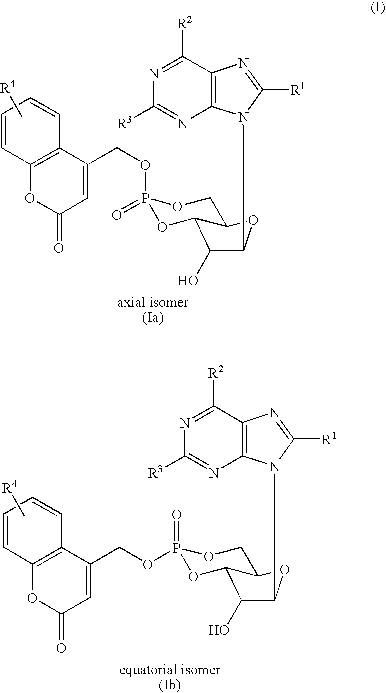
wherein
R¹ represents hydrogen, bromine or p-chlorophenylthio,
R⁴ represents 7-carboxymethoxy, 6,7-, 5,7- or 7,8-bis-(carboxymethoxy), 7-C₁-C₃-alkoxycarbonylmethoxy, 6,7-bis(C₁-C₃-alkoxycarbonylmethoxy), 5,7-bis(C₁-C₃-alkoxy-carbonylmethoxy), or 7,8-bis (C₁-C₃-alkoxycarbonylmethoxy),
R² represents —NH, and R³ is hydrogen (adenine residue), or
R² represents —OH and R³ represents —NH₂ (guanine residue),
   the compounds with the guanine residue (CGMP) being present in their dihydropyrimidone structure.

Preferred compounds are those wherein R⁴ represents carboxymethoxy or bis (carboxymethoxy). Particularly preferred are 6,7-bis(carboxymethoxy)- or 6,7-bis(C₁-C₃-alkoxycarbonylmethoxy)-substituted compounds.

In a preferred embodiment of the invention, C₁-C₃-alkoxy in the residue R⁴ represents a methoxy or ethoxy residue.

Particularly preferred compounds in the meaning of the invention are the following compounds 1-11:
1. [6,7-bis-(carboxymethoxy)coumarin-4-yl]methyl ester of cAMP
2. [7,8-bis-(carboxymethoxy)coumarin-4-yl]methyl ester of cAMP
3. [7-(carboxymethoxycoumarin-4-yl]methyl ester of cGMP
4. [6,7-bis(carboxymethoxy)coumarin-4-yl]methyl ester of cGMP
5. [6,7-bis(carboxymethoxy)coumarin-4-yl]methyl ester of 8-Br-cAMP
6. [6,7-bis(carboxymethoxy)coumarin-4-yl]methyl ester of 8-Br-cGMP
7. [5,7-bis(carboxymethoxy)coumarin-4-yl]methyl ester of 8-p-chlorophenylthio-cAMP
8. [6,7-bis(methoxycarbonylmethoxy)coumarin-4-yl]methyl ester of cAMP
9. [6,7-bis(ethoxycarbonylmethoxy)coumarin-4-yl]methyl ester of cGMP
10. [6,7-bis(carboxymethoxy)coumarin-4-yl]methyl ester of cGMP
11. [7-(carboxymethoxy)coumarin-4-yl]methyl ester of cAMP

According to the invention, the equatorial isomers of general formula Ib, especially those wherein R⁴ represents carboxymethoxy or bis(carboxymethoxy) are particularly preferred.

Surprisingly, the compounds of the invention exhibit a pattern of properties making them far superior to previously described caged cyclic nucleotides of the prior art when examining cyclic nucleotide-controlled cellular processes. All of the inventive coumarinylmethyl esters are extremely resistant to hydrolysis in aqueous buffer solutions, and the derivatives with free carboxymethoxy groups on the coumarin skeleton exhibit unexpectedly high solubility in aqueous buffer solutions, which is 10 to 25 times higher than that of comparable compounds of the prior art and sometimes >1,000 μM.

The compounds of the invention, inactive towards cAMP and cGMP binding sites, are photolabile and can be converted to the free, biologically active, native or 8-substituted cAMP derivatives and to the free, biologically active, native or 8-substituted cGMP derivatives in aqueous buffer solutions or after incorporation in a biological system by irradiation with UV light. Correspondingly substituted 4-hydroxy-methylcoumarins are formed as additional products of photolysis. Owing to the relatively high absorption at >300 nm and the comparatively high quantum yield, the efficiency of photo-liberation is good. In addition, the red-shift of the absorption maximum in the 6,7-disubstituted compounds by about 20 nm is particularly advantageous, allowing for efficient photolysis at up to 380 nm. Surprisingly, the free cyclic nucleotides are formed within a range of nanoseconds, i.e., extremely rapidly.

The compounds of the invention do not exhibit any prephotolytic biological activity and can be utilized with advantage in investigations on cell-free preparations and on cells peeled chemically or mechanically or on intact cells, e.g. using the patch-clamp technique.

The alkoxycarbonylmethoxy-substituted compounds of the invention exhibit good membrane-penetrating capacity and can be introduced into the cells by simple incubation. The photolabile coumarinylmethyl esters of the cyclic nucleotides, carboxymethoxy-substituted according to the invention and readily soluble in water, are liberated in the intracellular space by esterases present in the cell. Re-absorption is restricted as a result of the reduced membrane-penetrating capacity caused by the carboxylate groups, and higher intracellular concentrations of caged compounds can be obtained.

As a result of the low fluorescence of the inventive coumarinylmethyl esters compared to the high fluorescence of the substituted 4-hydroxymethylcoumarins formed in the photolysis, there is a massive increase in fluorescence during the photolysis, which correlates with the degree of photolysis and can be utilized in monitoring or quantifying the liberated cyclic nucleotide.

The new photolabile cGMP and cAMP esters can be used successfully in the investigation of cyclic nucleotide-controlled processes. Compared to the 4,5-dimethoxy-2-nitro-benzyl ester of cGMP, substantially smaller amounts of incident light at 333 nm on the [6,7-bis(carboxymethoxy)-coumarin-4-yl]methyl ester of cGMP result in a comparable activation of the olfactory cGMP-dependent cation channel and, while even a saturated solution of 7-methoxycoumarinylmethyl-caged cGMP in HEPES buffer will activate the olfactory cGMP-dependent cation channel not more than partially upon photolysis, the readily water-soluble compound of Example 3 easily achieves complete activation.

The invention therefore is also directed to the use of the inventive compounds in investigating cyclic nucleotide-dependent cellular processes in biological systems by measuring e.g. the cyclic nucleotide-controlled calcium influx into the cells.

On the other hand, the increase in fluorescence obtained by photolysis of the compounds according to the invention can be utilized for monitoring or quantifying the amount of liberated biologically active cyclic nucleotides cAMP, cGMP, 8-Br-cAMP, 8-Br-cGMP, 8-p-chlorophenylthio-cAMP, or 8-p-chlorophenylthio-cGMP. As a result of the low fluorescence of the coumarinylmethyl esters of formula I compared to the high fluorescence of the 4-hydroxymethylcoumarins formed in the photolysis, there is a massive increase in fluorescence during photolysis, which correlates with the degree of photolysis.

The compounds according to the invention can be used both as a mixture of isomers and in the form of their pure axial or equatorial isomers.

The compounds of the invention are prepared by reacting the free acids of cAMP, cGMP, 8-bromo-cAMP, 8-Br-cGMP, 8-p-chlorophenylthio-cAMP, or 8-p-chlorophenylthio-cGMP with the corresponding mono- or bis(lower alkoxycarbonylmethoxy)- or tert-butoxycarbonylmethoxy-substituted coumarin-4-yldiazomethanes, in which latter case the resulting tert-butoxycarbonylmethoxy-substituted coumarinylmethyl esters of the cyclic nucleotides, optionally after separation of the isomers, are converted to the corresponding carboxymethoxy-substituted coumarinylmethyl esters of the cyclic nucleotides using trifluoroacetic acid.

The substituted coumarinylmethyl esters of the cyclic nucleotides produced as a result of the alkylation reaction are obtained as a mixture of isomers comprised of the axial and equatorial forms. Purification of the crude products and—if necessary—separation of the mixture of isomers are performed using chromatography. To this end, the use of flash chromatography and/or HPLC was found to be particularly advantageous.

### EXAMPLES

### Example 1

### [6,7-Bis(carboxymethoxy)coumarin-4-yl]methyl ester of cAMP

A mixture of 1 mmol cAMP and 1.5 mmol 6,7-bis(tert-butoxycarbonylmethoxy)coumarin-4-yl]diazomethane (prepared from commercially available 6,7-dihydroxy-4-methylcoumarin by alkylation with tert-butyl bromoacetate, subsequent conversion to the 6,7-bis(tert-butoxycarbonylmethoxy)coumarin-4-carbaldehyde by oxidation with selenium dioxide, reaction of the aldehyde with p-toluenesulfonylhydrazine to form the corresponding p-toluenesulfonylhydrazone, and subsequent conversion to the diazo compound by treatment with triethylamine) in 8 ml DMSO and 32 ml of acetonitrile is stirred for 24 hours at 60° C. in the absence of light. The acetonitrile is removed on a rotatory evaporator and the DMSO and some byproducts are removed by repeated shaking with ether. The residue which includes the [6,7-bis(tert-butoxycarbonylmethoxy)coumarin-4-yl]methyl ester of cAMP is purified using flash chromatography (elution with chloroform/methanol) and/or preparative HPLC (PLRP-S, 10 μm, 250×25 mm i.d., flow rate 10 ml/min, linear gradient 20%-60% B for 70 min, eluant A: water, eluant B: acetonitrile) and optionally separated into the axial and equatorial isomers at the same time.

20 minutes of stirring performed with the pure axial or the pure equatorial form or with a mixture of both forms of the [6,7-bis(tert-butoxycarbonylmethoxy)coumarin-4-yl]methyl ester of cAMP in chloroform/TFA/water (25:74:1; about 2.5 ml per 20 mg) furnishes—after removal of the solvents and lyophilization—the [6,7-bis(carboxymethoxy)coumarin-4-yl]methyl ester of CAMP as the pure axial or pure equatorial isomer or as a mixture of the isomers.
Overall yield: 25% of theoretical amount.
Axial isomer: ³²P-NMR (DMSO-d₆): δ=−5.03;
   UV: λₘₐₓ (ε): 346 (12,000).
Equatorial isomer: ³²P-NMR (DMSO-d₆): δ=−3.26;
   UV: λₘₐₓ (ε): 347 (12,000).

### Example 2

### [7,8-Bis (carboxymethoxy)coumarin-4-yl]methyl ester of cAMP

In analogy to Example 1, using 1 mmol CAMP and 1.5 mmol [7,8-bis(tert-butoxycarbonylmethoxy)coumarin-4-yl]diazomethane (prepared from commercially available 7,8-hydroxy-4-methylcoumarin by alkylation with tert-butyl bromo-acetate, subsequent conversion to the 7,8-bis(tert-but-oxycarbonylmethoxy)coumarin-4-carbaldehyde by oxidation with selenium dioxide, reaction of the aldehyde with p-toluenesulfonylhydrazine to form the corresponding p-toluenesulfonylhydrazone, and subsequent conversion to the diazo compound by treatment with triethylamine).
Yield: 16%.
Axial isomer: ³¹P-NMR (DMSO-d₆): δ=−4.62;
   UV: λₘₐₓ(ε); 322.5 (11,500)
Equatorial isomer: ³¹ P-NMR (DMSO-d₆): δ=−3.18;
   UV: λₘₐₓ(ε): 321.5 (11, 000)

### Example 3

### 17-(Carboxymethoxy)coumarin-4-yl]methyl ester of cGMP

In analogy to Example 1, using 1 mmol B-Br-cGMP and 1.5 mmol [7-(tert-butoxycarbonylmethoxy)coumarin-4-yl]diazomethane (prepared from commercially available 7-hydroxy-4-methylcoumarin by alkylation with tert-butyl bromoacetate, subsequent conversion to the 7-tert-butoxycarbonyl-methoxycoumarin-4-carbaldehyde by oxidation with selenium dioxide, reaction of the aldehyde with p-toluenesulfonyl-hydrazine to form the corresponding p-toluenesulfonyl-hydrazone, and subsequent conversion to the diazo compound by treatment with triethylamine).
Yield: 15%.
Axial isomer: ³¹P-NMR (DMSO-d₆): δ=−5.08;
   UV: λₘₐₓ(ε): 326 (11,500).
Equatorial isomer.: ³¹P-NMR (DMSO-d₆): δ=−4.07;
   UV: λₘₐₓ(ε): 325 (11,200).

### Example 4

### [5,7-Bis(carboxymethoxy)coumarin-4-yl]methyl ester of cGMP

In analogy to Example 1, using 1 mmol cGMP and 1.5 mmol [5,7-bis(tert-butoxycarbonylmethoxy)coumarin-4-yl]diazo-methane (prepared from commercially available 5,7-dihydroxy-4-methylcoumarin by alkylation with tert-butyl bromoacetate, subsequent conversion to the 5,7-bis(tert-butoxycarbonylmethoxy)coumarin-4-carbaldehyde by oxidation with selenium dioxide, reaction of the aldehyde with p-toluenesulfonylhydrazine to form the corresponding p-toluenesulfonylhydrazone, and subsequent conversion to the diazo compound by treatment with triethylamine).
Yield: 33%.
Axial isomer: ³¹P-NMR (DMSO-d₆): δ=−5.26;
   UV: λₘₐₓ (ε): 324 (13,100).
Equatorial isomer: ³¹-P-NMR (DMSO-d₆): δ=−4.17;
   UV: λₘₐₓ (ε): 322 (13,300).

### Example 5

### [6,7-Bis(carboxymethoxy)coumarin-4-yl]methyl ester of 8-Br-cAMP

In analogy to Example 1, using 1 mmol 8-Br-cAMP and 1.5 mmol [6,7-bis(tert-butoxycarbonylmethoxy)coumarin-4-yl]diazomethane.
Yield; 16%.
Axial isomer: ³¹P-NMR (DMSO-d₆): δ=−4.68;
   UV: λₘₐₓ (ε): 347 (10,300) .
Equatorial isomer: ³¹P-NMR (DMSO-d₆): δ=−2.64;
   UV: λₘₐₓ (ε): 346 (10,000).

### Example 6

### [6,7-Bis(carboxymethoxy)coumarin-4-yl]methyl ester of 8-Br-cGMP

In analogy to Example 1, using 1 mmol 8-Br-cGMP and 1.5 mmol [6,7-bis(tert-butoxycarbonylmethoxy)coumarin-4-yl]diazomethane.
Yield: 25%.
Axial isomer: ³¹P-NMR (DMSO-d₆): δ=−4.96;
   UV: λₘₐₓ (ε): 349 (10,200)
Equatorial isomer: ³¹P-NMR (DMSO-d₆): δ=−3.96;
   UV: λₘₐₓ (ε): 346 (12,700).

### Example 7

### [5,7-Bis (carboxymethoxy)coumarin-4-yl]methyl ester of 8-p-chlorophenylthio-cAMP

In analogy to Example 1, using 1 mmol 8-pCPT-cAMP and 1.5 mmol [5,7-bis(tert-butoxycarbonylmethoxy)coumarin-4-yl]diazomethane.
Yield: 30%.
Axial isomer: UV: λₘₐₓ (ε): 322 (13,300).
Equatorial isomer: UV: λₘₐₓ (ε): 321 (13,300).

### Example 8

### [6,7-Bis(methoxycarbonylmethoxy)coumarin-4-yl]methyl ester of cAMP

In analogy to Example 1, using 1 mmol cAMP and 1.5 mmol (6,7-bis(methoxycarbonylmethoxy)coumarin-4-yl]diazomethane (prepared from commercially available 6,7-dihydroxy-4-methylcoumarin by alkylation with methyl bromoacetate, subsequent conversion to the 6,7-bis(methoxycarbonylmethoxy)-coumarin-4-carbaldehyde by oxidation with selenium dioxide, reaction of the aldehyde with p-toluenesulfonylhydrazine to form the corresponding p-toluenesulfonylhydrazone, and subsequent conversion to the diazo compound by treatment with triethylamine) and isolation and lyophilization of the two isomers directly following separation of the isomers by preparative HPLC.
Yield: 15%.
Axial isomer: ³¹P-NMR (DMSO-d₆): δ=−4.57;
   UV: λₘₐₓ (ε): 340 (10,700).
Equatorial isomer: ³¹P-NMR (DMSO-d₆): δ=−2.92;
   UV: λₘₐₓ (ε): 339 (10,800)

### Example 9

### [6,7-Bis(ethoxycarbonylmethoxy)coumarin-4-yl]3ethyl ester of cGMP

In analogy to Example 1, using 1 mmol cGMP and 1.5 mmol [6,7-bis(ethoxycarbonylmethoxy)coumarin-4-yl]diazomethane (prepared from commercially available 6,7-dihydroxy-4-methylcoumarin by alkylation with ethyl bromoacetate, subsequent conversion to the 6,7-bis(ethoxycarbonylmethoxy)-coumarin-4-carbaldehyde by oxidation with selenium dioxide, reaction of the aldehyde with p-toluenesulfonylhydrazine to form the corresponding p-toluenesulfonylhydrazone, and subsequent conversion to the diazo compound by treatment with triethylamine).
Yield: 25%.
Axial isomer: ³¹P-NMR (DMSO-d₆): δ=−4.99
   UV: λₘₐₓ (ε): 340 (11,000).
Equatorial isomer: ³¹P-NMR (DMSO-d₆): δ=−3.79;
   UV: λₘₐₓ (ε): 339 (11,000).

### Example 10

The compounds listed below were prepared as described in the preceding examples.
[5,7-Bis(carboxymethoxy)coumarin-4-yl]methyl ester of cAMP
[7-(Carboxymethoxy)coumarin-4-yl]methyl ester of cAMP
[6,7-Bis(ethoxycarbonylmethoxy)coumarin-4-yl]methyl ester of cAMP
[6,7-Bis(carboxymethoxy)coumarin-4-yl]methyl ester of cGMP
[7,8-Bis(carboxymethoxy)coumarin-4-yl]methyl ester of cGMP
[6,7-Bis(methoxycarbonylmethoxy)coumarin-4-yl]methyl ester of cGMP
[7-(Carboxymethoxy)coumarin-4-yl]methyl ester of 8-Br-cAMP
[5,7-Bis(carboxymethoxy)coumarin-4-yl]methyl ester of 8-Br-cAMP
[7,8-Bis(carboxymethoxy)coumarin-4-yl]methyl ester of 8-Br-cAMP
[6,7-Bis(methoxycarbonylmethoxy)coumarin-4-yl]methyl ester of 8-Br-cAMP
[6,7-Bis(ethoxycarbonylmethoxy)coumarin-4-yl]methyl ester of 8-Br-cAMP
[7-(Carboxymethoxy)coumarin-4-yl]methyl ester of 8 -Br-cGMP
[5,7-Bis(carboxymethoxy)coumarin-4-yl]methyl ester of 8-Br-cGMP
[7,8-Bis(carboxymethoxy)coumarin-4-yl]methyl ester of 8-Br-cGMP
[6,7-Bis(methoxycarbonylmethoxy)coumarin-4-yl] methyl ester of 8-Br-cGMP
[6,7-Bis(ethoxycarbonylmethoxy)coumarin-4-yl]methyl ester of 8-Br-cGMP
[7-(Carboxymethoxy)coumarin-4-yl] methyl ester of 8-p-chlorophenylthio-cAMP
[6,7-Bis(carboxymethoxy)coumarin-4-yl]methyl ester of 8-p-chlorophenylthio-cAMP
[7,8-Bis(carboxymethoxy)coumarin-4-yl]methyl ester of 8-p-chlorophenylthio-cAMP
[6,7-Bis(methoxycarbonylmethoxy)coumarin-4-yl]methyl ester of 8-p-chlorophenylthio-cAMP
[6,7-Bis(ethoxycarbonylmethoxy)coumarin-4-yl]methyl ester of 8-p-chlorophenylthio-cAMP
[7-(Carboxymethoxy)coumarin-4-yl]methyl ester of 8-p-chlorophenylthio-cGMP
[6,7-Bis(carboxymethoxy)coumarin-4-yl]methyl ester of 8-p-chlorophenylthio-cGMP
[5,7-Bis(carboxymethoxy)coumarin-4-yl]methyl ester of 8-p-chlorophenylthio- cGMP
[7,8-Bis(carboxymethoxy)coumarin-4-yl]methyl ester of 8 -p-chlorophenylthio-cGMP
[6,7-Bis(methoxycarbonylmethoxy)coumarin-4-yl]methyl ester of 8-p-chlorophenylthio-cGMP
[6,7-Bis(ethoxycarbonylmethoxy)coumarin-4-yl]methyl ester of 8-p-chlorophenylthio-cGMP

### Example 11

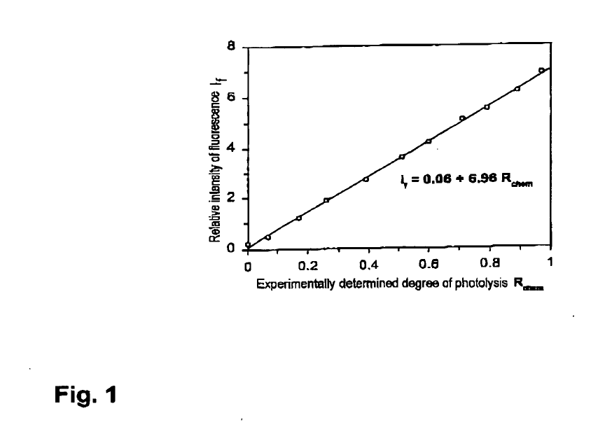

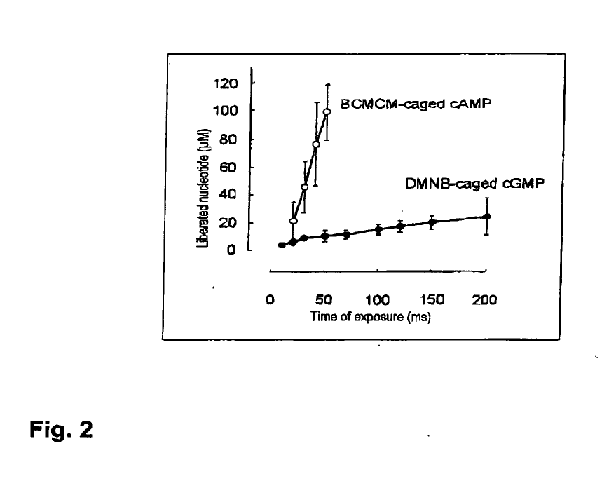

The intensity of fluorescence I_{f} as a function of the degree of photolysis R_{chem} in the photolysis of the axial form of the inventive [6,7-bis(carboxymethoxy)coumarin-4-yl]methyl ester of cGMP in HEPES buffer (λₒₓ=333 nm; λ_{f}=420 nm) is shown in FIG. 1

### Example 12

### Comparison of the Solubilities of Miscellaneous coumarinyl-methyl-caged Cyclic Nucleotides in Acetonitrile/HEPES Buffer (5:95) at pH 7.2 and Room Temperature

A suspension of the compounds in the specified solvent was treated for 5 min in an ultrasonic bath, undissolved components were removed by centrifugation, and the concentration in the solution was determined using HPLC. The following saturation concentrations were determined:

Prior Art Compounds:

(7-Methoxycoumarin-4-yl)methyl-caged cAMP (axial); 15 μM
(7-Methoxycoumarin-4-yl)methyl-caged cGMP (axial): 15 μM
(7-Methoxycoumarin-4-yl)methyl-caged cGMP (equatorial): 25 μM
(7-Methoxycoumarin-4-yl)methyl-caged 8-Br-cGMP (axial): 40 μM
(7-Methoxycoumarin-4-yl)methyl-caged 8-Br-cGMP (equatorial): 20 μM
   Compounds of the Invention:
[6,7-Bis(carboxymethoxy)coumarin-4-yl]methyl-caged cGMP (axial): >500 μM
[6,7-Bis(carboxymethoxy)coumarin-4-yl]methyl-caged cGMP (equatorial): >100μM
[6,7-Bis(carboxymethoxy)coumarin-4-yl]methyl-caged 8-Br-cGMP (axial): >500 μM
[6,7-Bis(carboxymethoxy)coumarin-4-yl]methyl-caged 8-Br-cGMP ( equatorial): >1000 μM
[7-(Carboxymethoxy)coumarin-4-yl]methyl-caged cAMP (axial). 900 μM
[7-(Carboxymethoxy)coumarin-4-yl]methyl-caged cAMP (equatorial): >1000 μM

### Example 13

### Activation of Cyclic Nucleotide-Dependent Ion Channels by Photolytic Liberation of Cyclic Nucleotides from the Corresponding Caged Compounds

Using whole cell measurements on HEK293 cells, the activation of the cyclic nucleotide-dependent ion channel CNCα3 (from olfactory cells) by photolytic liberation of cAMP from the axial form of the compound of Example 1 (BCMCM-caged AMP) and of the cyclic nucleotide-dependent ion channel CNCα2 (from rod photoreceptors) by photolytic liberation of cGMP from the axial form of 4,5-dimethoxy-2-nitrobenzyl ester of cGMP [DMN-caged cGMP, produced according to J. M. Nerbonne, S. Richard, J. Nargeot, H. A. Lester, Nature 310, 74 (1984)] was investigated comparatively by electrophysiological means. When using an intra-cellular solution of 500 μM of the compound of Example 1 and an intracellular solution of 200 μM of 4,5-dimethoxy-2-nitrobenzyl-caged cGMP and equal exposures to UV light (flashes of from 10 to 200 ms, 100 W Hg short-arc lamp equipped with a 335 nm cut-off filter), substantially higher concentrations of liberated cyclic nucleotide resulted for the compound of Example 1, as illustrated in the figure below.

1. New, photolabile coumarinylmethyl esters of cyclic nucleotides of general formula I 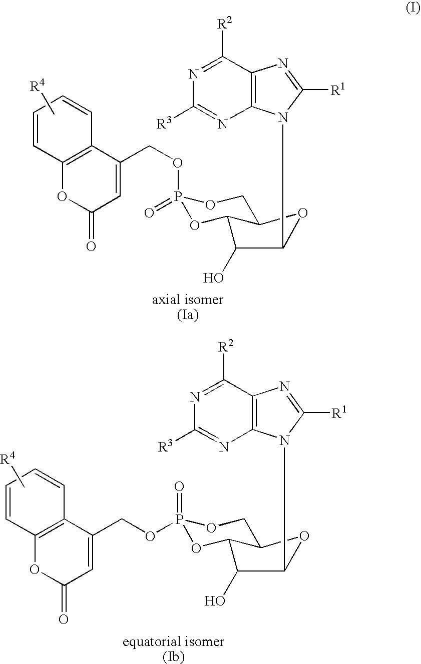
wherein
R¹ represents hydrogen, bromine or p-chlorophenylthio
R⁴ represents 7-carboxymethoxy, 6,7-, 5,7- or 7,8-bis(carboxymethoxy), 7-C₁-C₃-alkoxycarbonylmethoxy, 6,7-bis(C₁-C₃-alkoxycarbonylmethoxy), 5,7-bis(C₁-C₃-alkoxycarbonylmethoxy), or 7,8-bis(C₁-C₃-alkoxycarbonylmethoxy),
R² represents —NH₂ and R³ is hydrogen (adenine residue), or
R² represents —OH and R³ represents —NH₂ (guanine residue).

2. The compounds of general formula I according to claim 1,
characterized in that
R⁴ represents 7-carboxymethoxy, 6,7-, 5,7- or 7,8-bis(carboxymethoxy).

3. The compounds of general formula I according to claim 1,
characterized in that
R⁴ represents 6,7-bis(carboxymethoxy) or 6,7-bis(C₁-C₃-alkoxycarbonylmethoxy).

4. The compounds of general formula I according to claim 1,
characterized in that
C₁-C₃-alkoxy in residue R⁴ represents a methoxy or ethoxy residue.

5. The compounds of general formula I according to any of claims 1 to 4, comprising
1. [6,7-bis-(carboxymethoxy)coumarin-4-yl]methyl ester of cAMP
2. [7,8-bis-(carboxymethoxy)coumarin-4-yl]methyl ester of cAMP
3. [7-(carboxymethoxycoumarin-4-yl]methyl ester of cGMP
4. [6,7-bis(carboxymethoxy)coumarin-4-yl]methyl ester of cGMP
5. [6,7-bis(carboxymethoxy)coumarin-4-yl]methyl ester of 8-Br-cAMP
6. [6,7-bis(carboxymethoxy)coumarin-4-yl]methyl ester of 8-Br-cGMP
7. [5,7-bis(carboxymethoxy)coumarin-4-yl]methyl ester of 8-p-chlorophenylthio-cAMP
8. [6,7-bis(methoxycarbonylmethoxy)coumarin-4-yl]methyl ester of cAMP
9. [6,7-bis(ethoxycarbonylmethoxy)coumarin-4-yl]methyl ester of cGMP
10. [6,7-bis(carboxymethoxy)coumarin-4-yl]methyl ester of cGMP
11. [7-(carboxymethoxy)coumarin-4-yl]methyl ester of cAMP.

6. The compounds according to any of claims 1 to 5, comprising the equatorial isomers Ib.

7. A method of producing the new coumarinylmethyl esters of cyclic nucleotides of general formula I according to claim 1, wherein R⁴ represents 7-C₁-C₃-alkoxycarbonyl-methoxy, 6,7-bis(C₁-C₃-alkoxycarbonylmethoxy), 5,7-bis-(C₁-C₃-alkoxycarbonylmethoxy), or 7,8-bis(C₁-C₃-alkoxy-carbonylmethoxy),
characterized in that
cAMP, cGMP, 8-bromo-cAMP, 8-bromo-cGMP, 8-p-chlorophenylthio-cAMP, or 8-p-chlorophenylthio-cGMP are reacted with C₁-C₃-alkoxycarbonylmethoxy-substituted (coumarin-4-yl)diazomethanes, the mixture of isomers obtained is purified and optionally separated to furnish the isomers.

8. A method of producing the new coumarinylmethyl esters of cyclic nucleotides of general formula I according to claim 1, wherein R⁴ represents 7-carboxymethoxy, 6,7-, 5,7- or 7,8-bis(carboxymethoxy),
characterized in that
cAMP, cGMP, 8-bromo-cAMP, 8-bromo-cGMP, 8-p-chlorophenylthio-cAMP, or 8-p-chlorophenylthio-cGMP are reacted with tert-butoxycarbonylmethoxy-substituted (coumarin-4-yl)diazomethanes, the resulting tert-butoxy-carbonylmethoxy-substituted coumarinylmethyl esters are purified and optionally separated to yield the isomers, and the mixture of isomers or the isomers are subsequently treated with trifluoroacetic acid.

9. The method according to claim 7 or 8,
characterized in that
purification and separation of the isomers are performed using chromatography, preferably flash chromatography and/or HPLC.

10. Use of compounds according to claims 1 to 6 for investigating cyclic nucleotide-dependent cellular processes in biological systems.

11. Use of compounds according to claims 1 to 6 as biologically inactive photolabile precursors in the photochemical liberation of biologically active cAMP, cGMP, 8-Br-cAMP, 8-Br-cGMP, 8-p-chlorophenylthio-cAMP, or 8-p-chlorophenylthio-cGMP.

12. The use according to claim 10 or 11,
characterized in that
the compounds are employed as mixtures of isomers or in the form of their pure axial or pure equatorial isomers.
